(19) United States
(12) Patent Application Publication
(10) Pub. No.: US 2015/0011492 A1
(43) Pub. Date: Jan. 8, 2015

(54) ANTI-RESTENOSIS COMPOSITIONS AND METHODS

(71) Applicant: WISCONSIN ALUMNI RESEARCH FOUNDATION, Madison, WI (US)

(72) Inventors: F. Michael Hoffmann, Madison, WI (US); K. Craig Kent, Fitchburg, WI (US); Shakti Goel, Madison, WI (US); Lian-Wang Guo, Madison, WI (US); Toshio Takayama, Madison, WI (US)

(21) Appl. No.: 14/322,630

(22) Filed: Jul. 2, 2014

Related U.S. Application Data
(60) Provisional application No. 61/842,162, filed on Jul. 2, 2013.

(51) Int. Cl. A61K 31/704; A61F 2/82; A61K 31/517; A61K 31/436; A61K 31/047

(52) U.S. Cl. CPC A61K 31/704; A61K 31/436; A61K 31/047; A61K 31/517; A61F 2/82; A61F 2210/0061
USPC 514/34; 536/6.4; 623/1.2

(57) ABSTRACT

The present invention relates generally to the pathology of restenosis. In particular, provided herein are devices comprising a compound that selectively decreasing smooth muscle cell proliferation without a substantial decrease in endothelial cell proliferation. Also provided are methods of using such devices to treat, prevent, or reduce vascular disease or the likelihood of restenosis following angioplasty.

### CROSS-REFERENCE TO RELATED APPLICATIONS

This application claims the benefit of U.S. Provisional Patent Application No. 61/842,162, filed Jul. 2, 2013, which is incorporated herein by reference as if set forth in its entirety.

### STATEMENT REGARDING FEDERALLY SPONSORED RESEARCH OR DEVELOPMENT

This invention was made with government support under HL068673 awarded by the National Institutes of Health. The government has certain rights in the invention.

### FIELD OF THE INVENTION

The present invention relates generally to the pathology of restenosis. In particular, compositions, medical devices, and drug-eluting stents comprising a compound that selectively inhibits smooth muscle cell proliferation are provided herein. Also provided are methods of using compositions, medical devices, and drug-eluting stents of the present invention to treat, prevent, or reduce the likelihood of restenosis following angioplasty.

### BACKGROUND OF THE INVENTION

Atherosclerosis impedes blood flow to the heart, brain, other major organs and limbs, and is the leading cause of death in the United States. See, for review, Charo & Taub, Nature Reviews Drug Discovery 10:365-376 (2011). Treatments for occluded blood vessels associated with atherosclerosis include angioplasty, stenting, atherectomy, and bypass. Unfortunately, these widely implemented interventions are frequently associated with restenosis, a wound healing process that reduces the lumen diameter of a blood vessel due to scar tissue formation and which may ultimately result in reocclusion of the lumen. Despite the introduction of improved surgical techniques, devices, and pharmaceutical agents, restenosis occurs within six to twelve months after an angioplasty procedure in 25% to 50% of cases.

Restenosis, which is the leading cause of long-term failure of multiple forms of vascular reconstruction, involves two key processes: (1) neointimal hyperplasia resulting from the proliferation of smooth muscle cells (SMCs) when stimulated by growth factors, and (2) endothelial damage (e.g., vessel remodeling). SMCs and the matrix proteins they produce contribute to the development of hyperplastic plaques and, consequently, luminal narrowing, reduced blood flow, and vessel thrombosis. Endothelial damage commonly occurs at the time of or following intervention. To correct the problems associated with restenosis, additional revascularization procedures are frequently required, thereby increasing trauma and risk to the patient. Accordingly, there remains a need in the art for improved compositions and methods for treating atherosclerosis that are less likely to result in restenosis, reocclusion of a treated blood vessel, and delayed thrombosis.

### SUMMARY OF THE INVENTION

In a first aspect, the present invention is directed to a medical device comprising a formulation comprising between about 0.08 mg/mL and about 0.4 mg/mL idarubicin. The formulation can further comprise at least one of rapamycin, resveratrol, and halofuginone. The formulation can further comprise a pharmaceutically acceptable carrier. The pharmaceutically acceptable carrier can be a pluronic gel. The device can comprise a drug-eluting stent. In some cases, the device further comprises an expandable member having a first diameter for insertion into a vessel and a second diameter for making contact to walls of the vessel. A portion of the outer surface of the expandable member can comprise the idarubicin formulation. The expandable member can comprise a balloon.

In another aspect, the present invention provides a method for treating or preventing restenosis in a subject in need thereof. The method comprises following a vascular intervention that exposes the lumen of a blood vessel of the subject, providing to the lumen an effective amount of a compound capable of selectively decreasing smooth muscle cell proliferation without a substantial decrease in endothelial cell proliferation, where providing the effective amount of the compound treats or prevents restenosis in the blood vessel. In some cases, the compound is idarubicin or an analog thereof. The effective amount of idarubicin or an analog thereof can be between about 0.08 mg/mL and about 0.4 mg/mL. In some cases, idarubicin or an analog thereof is provided to the lumen in a formulation comprising at least 0.08 mg/mL idarubicin or an analog thereof. The idarubicin formulation can further comprise at least one of rapamycin, resveratrol, and halofuginone. The compound can be provided to the lumen using a medical device. In some cases, the medical device comprises a drug-eluting stent. The vascular intervention can be selected from the group consisting of an angioplasty, an atherectomy, a bypass surgical procedure, placement of a non-drug eluting stent, and placement of a drug-eluting stent. Treating or preventing restenosis can comprise reducing intimal hyperplasia.

In yet another aspect, the present invention provides a method for treating or preventing a vascular disease in a subject in need thereof. The method comprises providing to the lumen of a blood vessel of the subject an effective amount of a compound capable of selectively decreasing smooth muscle cell proliferation without a substantial decrease in endothelial cell proliferation, whereby vascular disease in the subject is treated or prevented. In some cases, the vascular disease is atherosclerosis. The compound can be idarubicin or an analog thereof. The effective amount of idarubicin or an analog thereof can be between about 0.08 mg/mL and about 0.4 mg/mL. Idarubicin or the analog thereof can be provided to the lumen in a formulation comprising at least 0.08 mg/mL idarubicin. In some cases, the idarubicin formulation further comprises at least one of rapamycin, resveratrol, and halofuginone. The compound can be provided to the lumen using a medical device. The medical device can comprise a drug-eluting stent. In some cases, the subject has undergone or will undergo a vascular intervention. The vascular intervention can be selected from the group consisting of an angioplasty, an atherectomy, a bypass surgical procedure, placement of a non-drug eluting stent, and placement of a drug-eluting stent. In some cases, treating or preventing a vascular disease comprises reducing intimal hyperplasia.

### BRIEF DESCRIPTION OF THE DRAWINGS

FIGS. 1A-B present data from an AlamarBlue® cell viability assay using resveratrol. Experiments were performed as described in Example 1 (below). DMSO (40 wells) and resveratrol (40 wells) were used as negative control and positive control, respectively. Data are presented either as (A) level of AlamarBlue® fluorescence from individual wells (A); (B) a mean±SD (standard deviation) of 40 wells (B) (***P<0.001).

FIG. 2 presents data from a CellTiter-Glo test assay using rapamycin.

FIGS. 3A-B present data from the NIH Clinical Collection pilot screen for HuAoSMC proliferation. Assays were performed with SMCs using the automated assay system as described in the Examples. DMSO (blue, final 0.05% in each of 8 wells) and resveratrol (red, final 50 mM in each of 8 wells) served as negative control and positive control, respectively, on each of six 96-well plates. Total 447 compounds in the NIH Clinical Collection (yellow) were tested at a final concentration of 5 mM (1 well for each drug). (A) Percent AlamarBlue® cell viability fluorescence. The dashed line marks 50% inhibition of SMC proliferation. (B) Consistency of HTS assay for each of six 96-well plates.

FIG. 4 illustrates confirmation of eight hits (50-90% inhibition) from the NIH clinical library with CellTiter-Glo. Blue: AlamarBlue®. Black: CellTiter-Glo (not available for 7 and 8). Note that halofuginone is not included in the NIH Clinical Library.

FIGS. 5A-B present comparative dose responses of HuAoSMCs and HuAoECs for idarubicin (A) and resveratol (B). Proliferation of SMCs or ECs in the presence of various concentrations of idarubicin or resveratrol was assayed in 96-well plate. Each data point is a mean 6 SD of triplicates, *P<0.05.

FIGS. 6A-E illustrate that halofuginone inhibits intimal hyperplasia but not re-endothelialization following angioplasty. DMSO control (A, D) or halofuginone (30 μg) (B, E) dissolved in 200 μL pluronic gel was applied around the injured rat carotid artery. Rats were sacrificed at 14 days post injury, and carotid sections were prepared for H&E staining (A, B) or CD31 staining (D, E). The intima/media ratio was quantified (C). Each bar represents a mean μ SEM of 5 rats. EEL, external elastic lamina. IEL, internal elastic lamina.

FIGS. 7A-C demonstrate that local administration of idarubicin in a rat carotid angioplasty model effectively impairs intimal hyperplasia but not re-endothelialization. (A and B) While idarubicin effectively reduced intimal hyperplasia (intima/media), the integrity of the endothelial layer (re-endothelialization) of the injured vessel was not affected (C) (CD31 staining) Idarubicin inhibits intimal hyperplasia but preserves re-endothelialization following angioplasty.

FIG. 8 presents a time-course of cumulative idarubicin release from a PLGA membrane. (p<0.05) (n=3).

FIGS. 9A-E present demonstrates the inhibitory effect of idarubicin on intimal hyperplasia in balloon-injured rat carotid arteries. Following balloon angioplasty, idarubicin was applied locally around the injured arteries. Morphometric analysis was performed on the sections of carotid arteries collected on day 14 post angioplasty. Shown in A and B are representative H&E-stained sections from the arteries treated with vehicle (DMSO) and idarubicin, respectively. Arrow heads point to IEL. Statistics of the area ratio of intima versus media (C), residual lumen (the ratio of lumen area versus IEL area) (D), and EEL length (E) were calculated with the data pooled from 5 rats in each treatment group. Each bar represents a mean±SEM (*p<0.05).

FIGS. 10A-D demonstrate the absence of idarubicin's effect on re-endothelialization in balloon-injured rat carotid arteries. Following balloon angioplasty, idarubicin was applied locally around the injured arteries. For determination of re-endothelialization, immunostaining of CD31 was performed on the sections of carotid arteries collected on day 14 post angioplasty. Shown in A and B are representative immunostained sections from the arteries treated with vehicle (DMSO) and idarubicin, respectively. Arrow heads point to IEL. A section of uninjured right carotid artery (C) shows CD31 staining of the undisrupted endothelial layer (see the brown circle). The relative score of re-endothelialization (stained versus total circumference) was quantified with the data pooled from 5 rats in each treatment group (D). Each bar represents a mean±SEM.

### DETAILED DESCRIPTION OF THE INVENTION

The present invention is directed to compositions and methods of using such compositions to treat or prevent restenosis following, for example, angioplasty. The present invention is based, at least in part, on the Inventors' discovery that a rat carotid angioplasty model of restenosis is useful for identifying candidate compounds having the potential to treat or prevent restenosis. Through experiments described in the Examples below, the Inventors found that a drug used for the treatment of leukemia had an inhibitory effect on smooth muscle cell (SMC) proliferation but did not negatively affect endothelial cell (EC) proliferation. From dose-response assays, the Inventors further discovered that idarubicin inhibits intimal hyperplasia in a dose-dependent manner but preserves and promotes re-endothelialization following angioplasty. This finding is particularly important given that traditional therapeutic methods for atherosclerosis are frequently associated with damage to the endothelium, a sheet of endothelial cells forming an antithrombotic inner lining of blood vessels—damage that often reduces blood flow to pre-angioplasty levels and induces endothelial cells to create a pro-thrombotic microenvironment. Integrity of the endothelium is essential for vascular homeostasis and blood flow. Likewise, maintenance and restoration of the endothelium is integral to treating and preventing restenosis.

Compositions

Accordingly, in one aspect, the present invention provides compositions useful for treating or preventing restenosis. In an exemplary embodiment, a composition according to the present invention can be a medical device. As used herein, the term “medical device” refers to a device or other composition which can be employed to perform a minimally invasive operation within a lumen of a blood vessel. For example, a medical device can be a vessel expansion unit such as, without limitation, a dilation balloon, stent delivery system, balloon-expanding stent, self-expending stent, a substance delivery unit such as a coated or drug-eluting stent, a coated or drug-eluting bio-absorbable stent, a drug delivery balloon, a percutaneous valve system, a percutaneous coronary intervention (PCI) device, an ablation unit (e.g., a laser, cryogenic fluid unit, electric impulse unit, cutting balloon, rotablator, directional atherectomy unit, or transluminal extraction unit), a brachytherapy unit, or guidewire.

In some cases, a composition can be an implantable medical device for delivering a therapeutic agent within a lumen such as, for example, a blood vessel. As used herein, the term “lumen” refers to an organic tubular structure of a subject (e.g., a human patient) such as, without limitation, an artery, vein, cardiac vessel, brain vessel, part of the urogenital system, nephrotic system, hepatic system, or bronchus tree.

As used herein, the term “agent” refers to any therapeutic agent or drug, as well as any body analyte, such as glucose. The terms “drug” and “therapeutic agent” are used interchangeably to refer to any therapeutically active substance that is delivered to a bodily lumen of a living subject to produce a desired effect (e.g., a beneficial effect) therein. In some cases, a composition provided herein can be configured to deliver an agent that produces a desired effect within the lumen. In particular, a desirable effect produced upon delivery of an agent within a lumen can include, without limitation, reduced or prevented intimal hyperplasia, selectively reduced or inhibited smooth muscle cell proliferation, increased re-endothelialization, increased endothelial cell viability, increased endothelial cell proliferation, and reduced or inhibited delayed stent thrombosis (i.e., blood clotting). For example, a composition according to the present invention can be an implantable medical device for delivering an anti-restenosis agent to a lumen.

In one embodiment, a therapeutic agent provided or delivered by a medical device of the present invention comprises a formulation of idarubicin or an analog thereof. Idarubicin, or (7S,9S)-9-Acetyl-7-[(3-amino-2,3,6-trideoxy-α-L-lyxo-hexapyranosyl)oxy]-7,8,9,10-tetrahydro-6,9,11-trihydroxy-5,12-naphtacenedione, is an antineoplastic drug useful for the treatment of acute myeloid leukemia (AML) in adults. An analog of idarubicin useful for the inventions provided herein can be the 5-deoxypyranoanthracycline analog of idarubicin that was synthesized using a convergent and regioselective synthesis protocol as described by LavaHee et al., Synthesis of a 5-deoxypyranoanthracycline: An entry into novel analogs of idarubicin, Tetrahedron Lett. 34(22):3519-3522 (1993).

As demonstrated in the Examples, idarubicin exhibits enhanced selectivity for impairing smooth muscle cell proliferation without affecting endothelial cell proliferation as compared to anti-restenosis agents presently used in drug-eluting stents. These agents include rapamycin and paclitaxel. Relative to controls not contacted to a formulation comprising idarubicin, idarubicin preferentially inhibits up to 80% of smooth muscle cell proliferation while inhibiting less than 40% of endothelial cell proliferation. Idarubicin hydrochloride is distributed by Pfizer under the brand name Idamycin®. Generic idarubicin hydrochloride is available from various manufacturers including Teva Pharmaceuticals USA.

A formulation of idarubicin or an analog thereof can comprise about 0.08 mg/mL to about 0.4 mg/mL idarubicin (in pluronic gel). In some cases, the formulation further comprises one or more additional agents. For example, an idarubicin formulation can additionally comprise halofuginone, rapamycin, or resveratrol. Appropriate amounts of such additional agents include about 0.1 mg/mL halofuginone (dissolved in pluronic gel), about 0.1 to about 1.0 mg/mL rapamycin (dissolved in pluronic gel), and about 2.2 mg/mL resveratrol (dissolved in pluronic gel).

A medical device according to the present invention can be a stent. As used herein, the term “stent” refers to a mesh tube that is inserted into a blood vessel using a catheter to prop open the vessel and prevent it from collapsing. Unlike angioplasty, in which an expandable member (e.g., a balloon) is inflated to push aside a blockage and then is removed from the subject's blood vessel, a stent is permanently implanted in the subject's blood vessel. A stent comprising a formulation of idarubicin or an analog thereof is useful for a variety of medical procedures including, by way of non-limiting example, treatment of occluded regions of blood vessels caused by abnormal or inappropriate migration and proliferation of smooth muscle cells, thrombosis, and restenosis. A stent according to the present invention can be placed in any appropriate blood vessel including both arteries and veins. For example, a stent can be placed in the aorta or the iliac, femoral, renal, or coronary artery (for percutaneous transluminal coronary angioplasty).

In exemplary embodiments, a medical device of the present invention is a drug-coated stent, also called a drug-eluting stent. Unlike bare metal stents, drug-coated stents have special drug coatings that release compounds over time to reduce the incidence of reocclusion. While drug-eluting stents have been generally effective for reducing rates of restenosis, certain types of atherosclerotic plaques cannot be treated with stents. In addition, standard stents are frequently associated with incomplete stent apposition, re-endothelialization and/or abnormal endothelial cell function, all of which can contribute to late stent thrombosis and, in some cases, increased mortality.

In an exemplary embodiment, a medical device provided herein can be employed for a subject after the subject undergoes a therapeutic or preventative vascular procedure. Such vascular procedures can include, without limitation, an angioplasty, an atherectomy, a bypass surgical procedure, or a procedure to place a non-drug eluting (i.e., bare metal) stent or a drug-eluting stent. Angioplasty, which is the inflation of a balloon inside a blood vessel to open a blockage, can be performed alone or in conjunction with placement of a stent, if necessary, to keep the artery open.

In some cases, a medical device of the present invention is a drug-coated or drug-eluting inflatable device (e.g., balloon) suitable for use with angioplasty or another anti-restenosis therapeutic procedure. Drug-eluting angioplasty balloons have been shown to provide local drug effects. See, for example, Agostoni et al., JACC Cardiovasc. Interv., available online 15 May 2013, ISSN 1936-8798. Accordingly, a medical device of the present invention can include a drug-eluting or drug-coated inflatable device comprising a formulation of idarubicin or an analog thereof

Medical devices of the present invention can be fabricated from biocompatible, metallic, ceramic, polymeric, or composite materials, or a combination thereof. Where a medical device of the present invention is fabricated from polymeric materials, suitable polymers can be biostable and, in some cases, biodegradable.

For coatings comprising one or more active agents, the agent will be retained on the medical device during delivery and expansion of the device, and released at a desired rate and for a predetermined duration of time at that position within the blood vessel. For example, a stent coated by a formulation comprising idarubicin or an analog thereof and, in some cases, one or more additional agents, can be expanded at a desired position for treatment.

Medical devices comprising an idarubicin formulation as provided herein can be used to treat a subject having a condition or disorder that requires a treatment. Such a subject can be treated by, for example, implanting a medical device described herein in a blood vessel of a subject. Preferably, the subject is a human being. Exemplary disorders or conditions that can be treated by the method disclosed herein include, without limitation, thrombosis, hemorrhage, vascular dissection or perforation, vascular aneurysm, vulnerable plaque, chronic total occlusion, anastomotic proliferation for vein and artificial grafts, tumor obstruction, restenosis and progression of atherosclerosis in patient subpopulations including subpopulations having Type I diabetes, Type II diabetes, metabolic syndromes, vulnerable lesions, systemic infections, and combinations thereof. Medical devices including drug-coated stents provided herein can be used for, or in conjunction with, any appropriate medical procedure. For example, delivery or placement of a medical device provided herein can be accomplished using methods well known to those skilled in the art.

Suitable pharmaceutical carriers, vehicles and diluents for formulations described herein include, without limitation, inert solid diluents or fillers, sterile aqueous solutions and various organic solvents. For example, a pharmaceutically acceptable carrier, vehicle, or diluent can be a pluronic gel or other nonionic surfactant.

Methods

The present invention also provides methods for treating or preventing restenosis. The methods provided herein are particularly suited to preventing or reducing the incidence of restenosis following angioplasty. Angioplasty ruptures and dissects plaques from the underlying vessel layer and partially dissects the intima and media. See, e.g., Krueger et al., Radiology 231:546-554 (2004). Angioplasty often damages endothelial cells and collagen fibrils in the subendothelial layer and overstretches the vessel wall as well. Damage to these tissues activates an intrinsic coagulation pathway and triggers smooth muscle cell proliferation and migration into the subintimal space of the vessel.

Accordingly, in one aspect, the present invention provides a method for treating or preventing restenosis in a subject. The method comprises providing an amount of compound that is effective for selectively decreasing smooth muscle cell proliferation without a substantial decrease in endothelial cell proliferation.

As used herein, the terms “treat” and “treating” refer to improving, reducing, eliminating, mitigating, or lessening the severity of any aspect of restenosis in a subject. In some cases, treating restenosis can include selectively reducing smooth muscle cell proliferation at or near the site of treatment, maintaining or increasing endothelial cell proliferation or migration at or near the site of treatment, reducing mortality, reducing or preventing reocclusion of a treated vessel, and promoting survival in a subject receiving or previously subjected to a therapeutic or preventative vascular procedure (e.g., angioplasty).

As used herein, the terms “prevent” and “preventing” refer to limiting, diminishing, mitigating, or lessening the onset, occurrence, development of any aspect of restenosis in a subject. In an exemplary embodiment, preventing restenosis in a subject can comprise taking proactive or prophylactic measures to eliminate or reduce the risk of restenosis occurring in the subject. In some cases, a method for preventing restenosis in a subject comprises providing an effective amount of a compound capable of selectively decreasing smooth muscle cell proliferation without a substantial decrease in endothelial cell proliferation. Preventing restenosis can also comprise providing an effective amount of such a compound to a subject prior to or following treatment of the subject using a therapeutic or preventative vascular procedure (e.g., angioplasty).

In some cases, methods for treating or preventing restenosis can include reducing intimal hyperplasia. As used herein, the term “intimal hyperplasia” refers to a proliferative response in intimal smooth muscle cells. Intimal smooth muscle cell proliferation can produce multi-layered plaques comprising cells that express alpha-smooth-muscle actin. See, e.g., Stary et al., Arterioscler. Thromb. 12:120-134 (1992). Accordingly, a method of the present invention can comprise reducing or preventing an intimal proliferative response after angioplasty. In other cases, treating or preventing restenosis can include reducing or preventing arterial remodeling, which includes changes in artery wall geometry such as a change in the total circumference of an artery.

In an exemplary embodiment, a method provided herein is performed for a subject prior to or after the subject undergoes a therapeutic or preventative vascular procedure. Such vascular procedures can include, without limitation, an angioplasty, an atherectomy, a bypass surgical procedure, or a procedure to place a non-drug eluting (i.e., bare metal) stent or a drug-eluting stent. Angioplasty, which is the inflation of a balloon inside a blood vessel to open a blockage, can be performed alone or in conjunction with placement of a stent, if necessary, to keep the artery open. In some cases, therefore, a method for reducing or preventing restenosis is performed for a subject previously subjected to a vascular procedure such as a surgical bypass procedure. For example, a drug-eluting stent comprising an effective amount of an idarubicin formulation can be provided following or in conjunction with a surgical bypass procedure. Such a drug-eluting stent can have increased efficacy for treating or preventing restenosis relative to a standard bare metal or drug-eluting stent when used following or in conjunction with surgical bypass.

In other cases, a method for treating or preventing restenosis is performed for a subject previously subjected to a vascular procedure such as angioplasty of a peripheral artery. Such a drug-eluting stent can have increased efficacy for treating or preventing restenosis relative to a standard bare metal or drug-eluting stent when used following or in conjunction with angioplasty of peripheral arteries.

An effective amount of compound for treating or preventing restenosis or any aspect of restenosis can be an amount of a compound to yield a selective decrease in smooth muscle cell proliferation but not a substantial or significant decrease in endothelial cell proliferation. For example, an effective amount of idarubicin or an analog thereof can be about 0.08 mg/mL to about 0.4 mg/mL idarubicin (in pluronic gel). In some cases, the formulation further comprises one or more additional agents such halofuginone, rapamycin, or resveratrol. Appropriate amounts of such additional agents include about 0.1 mg/mL halofuginone (dissolved in pluronic gel), about 0.1 to about 1.0 mg/mL rapamycin (dissolved in pluronic gel), and about 2.2 mg/mL resveratrol (dissolved in pluronic gel).

Efficacy of a compound for treating or preventing restenosis can be determined using any appropriate method. Smooth muscle cell proliferation can be quantified by counting cells positive for Ki-67 or another nuclear protein. Endothelial cell proliferation can be quantified by detecting cells positive for CD31 or another specific endothelial marker. CD31 is a 130 kDa endothelial cell adhesion molecule that was initially identified from ECs and platelets. See van Mourik, J. Biol. Chem. 260:11300-11306 (1985). For example, CD31⁺ endothelial cells can be detected in paraffin-embedded sections of small or large vessels (e.g., carotid sections). To detect a selective decrease in smooth muscle cell proliferation without a substantial decrease in endothelial cell proliferation, the detection and quantification methods described herein can be used to determine the ratio of stained perimeter to the total perimeter of an endothelial layer in, for example, an arterial cross-section. For example, appropriate detection and quantification methods can be used to determine what percentage of the total perimeter measurement is CD31⁺ luminal perimeter. In some cases, it can be advantageous to assign a score (from 1 to 5) to the percentage of CD31⁺ luminal perimeter. For example, score of 1 can be assigned for 0% to about 20% endothelialization; 2=about 20% to about 40% endothelialization; 3=about 40% to about 60%; 4=about 60% to about 80%; and 5=about 80% to 100%.

The methods provided herein can be used for or in conjunction with any appropriate medical procedure. For example, providing an effective amount of a compound described herein can include delivery or placement of a medical device comprising the compound in conjunction with one or more medical procedures (e.g., echocardiogram, cardiac angiography) well known to those skilled in the art.

Unless defined otherwise, all technical and scientific terms used herein have the same meaning as commonly understood by one of ordinary skill in the art to which the invention pertains. While the invention has been described in detail with reference to preferred methods and materials, it will be apparent to one of ordinary skill in the art that various changes and modifications can be made and equivalents employed, without departing from the present invention.

The invention will be more fully understood upon consideration of the following non-limiting Examples. All papers and patents disclosed herein are hereby incorporated by reference as if set forth in their entirety.

### EXAMPLES

### Example 1

### Materials & Methods for Cell Proliferation Assays

Alamar Blue was purchased from Invitrogen (Carlsbad, Calif.). Cell Titer Glo was from Promega (Madison, Wis.). Primary human aortic smooth muscle cells (HuAoSMCs) and primary human aortic endothelial cells (HuAoECs) at passage 3 were purchased from Lonza; their respective optimal culture media (SmGM-2 and EGM-2) were from the same commercial source. Cells were used at passage 5 after expansion. Trypsin/EDTA solution was from Clonetics (Walkersville, Md.); and DPBS was from Gibco (Invitrogen, Carlsbad, Calif.). Microtiter tissue 96-well culture plates with transparent flat-bottoms and black-walled sides were from Costar (Corning, N.Y.). Resveratrol and idarubicin were products of Sigma-Aldrich (St. Louis, Mo.). Stock solutions of these reagents were prepared in DMSO (Thermo-Fisher). The library of NIH Clinical Collection composed of 447 unique compounds of known bioactivity was available at the Small Molecule Screening and Synthesis Facility (SMSSF) of the University of Wisconsin Carbone Cancer Center (UWCCC).

Cryo-protected frozen HuAoSMCs and HuAoECs (Lonza, passage 3) were thawed and cultured in their respective media that are optimized for cell growth by the manufacturer. HuAoSMCs (“SMCs”) were grown in the SmGM-2 medium containing 5% FBS, and HuAoECs (“ECs”) in the EGM-2 medium containing 2% FBS in a humidified incubator at 37° C. with 5% CO₂. After expansion, cells at passage 5 were used for all the experiments.

Conditions that would produce favorable and consistent outcomes for high-throughput screening of compounds affecting human aortic smooth muscle cell (SMC) and endothelial cell (EC) proliferation were determined. Factors such as cell passage, seeding density, serum concentration, the addition of growth factors, and assay duration were varied. For human SMCs, cell densities ranging from about 1,000 cells per well to about 5,000 cells per well were aliquoted into 96-well plates and cultured in the SmGM-2 medium (Lonza CC-3182) with 5% serum. Cells seeded with 3000/well had the most rapid growth rate. Accordingly, this seeding density was selected for later experiments. A similar protocol was developed for ECs, which were grown in EGM-2 medium containing 2% serum.

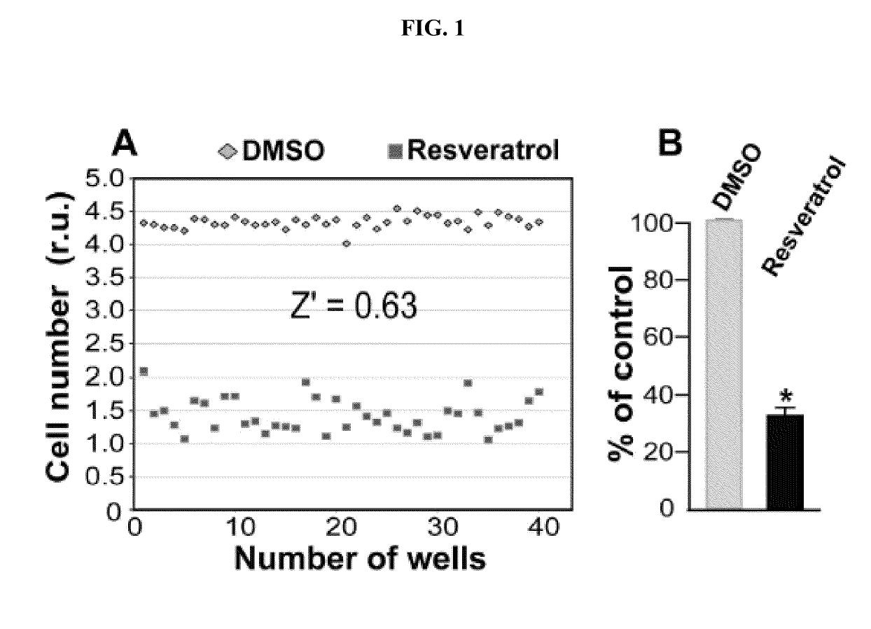

Using the determined seeding and growth conditions, control experiments were performed to test the consistency of the automated high-throughput screening (HTS) system using Resveratrol as a control drug (FIG. 1). Resveratrol was chosen because this natural compound inhibits angioplasty-induced intimal hyperplasia. See Breen et al., Atherosclerosis 222:375-381 (2012); Kim et al., Molecular Nutrition & Food Res. 54:1497-1505 (2010).

Freshly collected HuAoSMCs (passage 5) were counted (<93% viability) by Cellometer AutoT4 (Nexelon Bioscience), and dispensed using Microflo Select (BioTek) to a final density of 2700 cells/200 ml/well in the SmGM-2 medium in a 96-well plate. After a 24 hour incubation to allow cell attachment, 0.1 ml of DMSO (vehicle) or 0.1 ml of resveratrol (a known SMC growth inhibitor) stock in DMSO was robotically transferred using Biomek FX (Beckman) from a resveratrol stock plate into cell culture (final 50 mM resveratrol). DMSO and resveratrol were added into alternate columns of wells (8 wells per column). Cell growth/proliferation was monitored by reading fluorescence from the AlamarBlue® cell viability reagent (Invitrogen). See Antczak et al., J. Biomol. Screening 12:521-535 (2007); Nociari et al., J. Immunological Methods 213:157-67 (1998). After incubation with resveratrol for 72 hours, AlamarBlue reagent was added using Matrix Hydra (Thermo-Fisher) and incubated with cells for another 24 hours, and fluorescence was then determined using a Safir2 plate reader (Tecan, excitation/fluorescence: 530 nm/590 nm, bandwidth: 15 nm). The data from 40 wells of vehicle and 40 wells of resveratrol treatments were analyzed for assessment of well-well consistency in the assay. Background signal from the cell-free wells (medium only) was subtracted. It was determined that reading Alamar Blue fluorescence 24 hours after incubation reduced variance compared to reading after shorter incubation (e.g., 4 hours). To verify assay consistency with a additional method, AlamarBlue® was removed and wells were gently washed, and Cell Titer Glo reagent was then added followed by a 10 minute incubation and luminescence measured using Genios Pro.

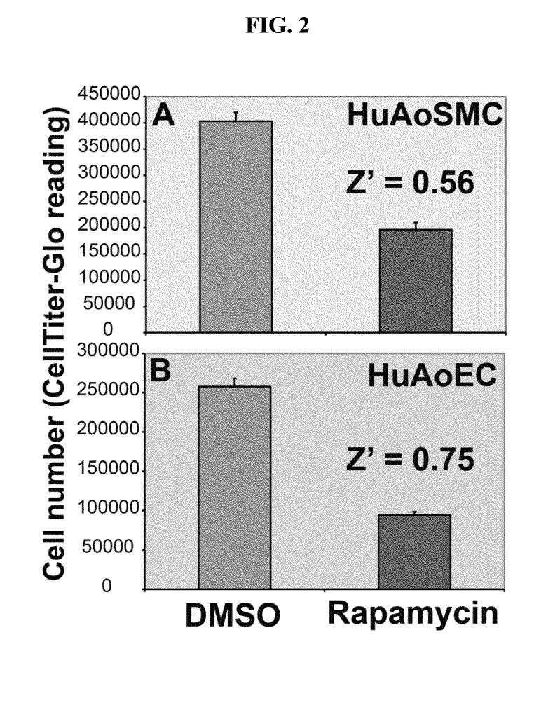

Resveratrol significantly inhibited human SMC growth by approximately 65%, with a very small SEM, indicating low well-to-well variation, as compared to data for untreated controls in DMSO (FIGS. 1A-B). This robust well-to-well consistency achieved by our experimental system is better reflected by a Z′ value of 0.63. The Z′ value is generally accepted as a measure to quantify the quality and, hence, suitability of a particular assay for use in a full-scale, high-throughput screen. See, e.g., Tomasini-Johansson et al., J. Intl. Soc. Matrix Biol. (2012); Zhang et al., J. Biomol. Screening 4:67-73 (1999); Patel et al., PloS One 7:e36594 (2012). In parallel experiments, a CellTiter-Glo® luminescent cell viability assay (Cat. #7570, Promega), which quantifies ATP levels in cell lysates, was evaluated as an additional and more sensitive orthologous assay for cell number. Sachsenmeier et al., PLoS One 17:993-8 (2012). Using the CellTiter-Glo® assay, Rapamycin which is used in drug-eluting stents at 200 nM produced >50% inhibition of both human SMCs and ECs with Z′ values of 0.56 to 0.75 respectively (FIG. 2). In addition, CellTiter-Glo® assays using resveratrol for both SMCs and ECs also produced high Z′ values (>0.7) (data not shown). Curve fitting was performed with the Prism software (GraphPad).

### Example 2

### Preliminary Screen of a NIH Clinical Library on Human SMCs and ECs

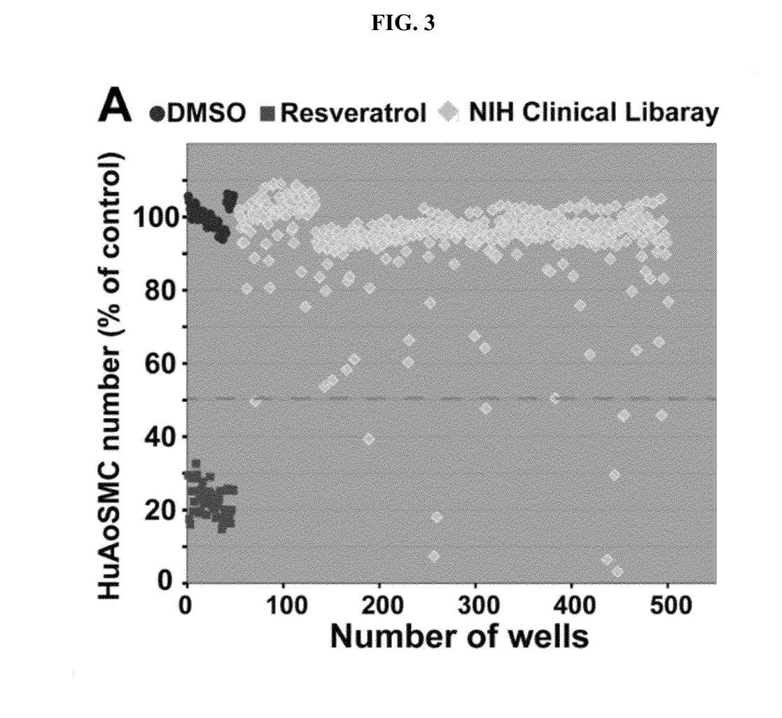

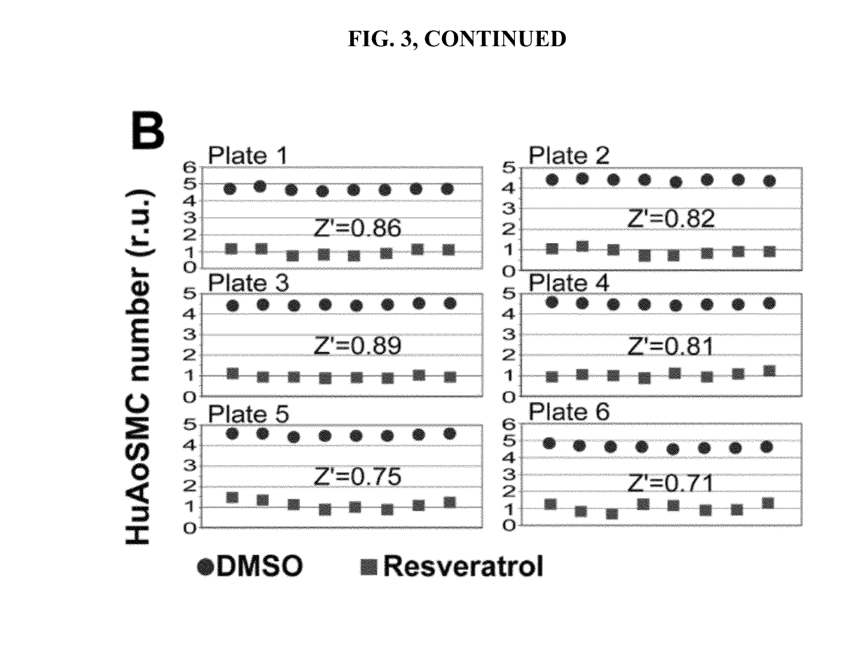

In search of such selective drugs, we have developed robust high throughput screening (HTS) assays for human aortic SMC and EC proliferation (Z′>0.7) using a robotic system at the UW Small Molecule Screening & Synthesis Facility (available at hts.wisc.edu on the World Wide Web). A preliminary screen of a 447-compound NIH Clinical Library was performed using a compound concentration of 5 μM in six 96-well plates with eight wells of negative control (vehicle, DMSO) and eight wells of positive control (resveratrol) per plate (FIG. 3A). The assay was performed as described above. Briefly, human SMCs were suspended at a density of 3000 cells/200 μL medium/well and added to 96-well plates. NIH library compounds in dimethyl sulfoxide (DMSO) or vehicle-alone were administered to cells at a final concentration of 5 μM and incubated for 3 days. AlamarBlue® cell viability reagent (20 μL) was then added and, after a 24-hour incubation, fluorescence was measured. The overall signal to background ratio was 5.1±0.4 for all plates. A Z′ value for each individual plate was calculated using the mean and SD from the negative and positive controls. All of the Z′ values were in the range of 0.71-0.89 (FIG. 3B). The overall Z′ value calculated with the pooled data from all six plates was 0.73. Thus, high well-to-well and plate-to-plate consistencies were observed.

All data are presented as mean 6 standard error (SEM). Statistical analysis was performed using two-tailed unpaired Student's t-test. Data were considered statistically significant when a P value is <0.05.

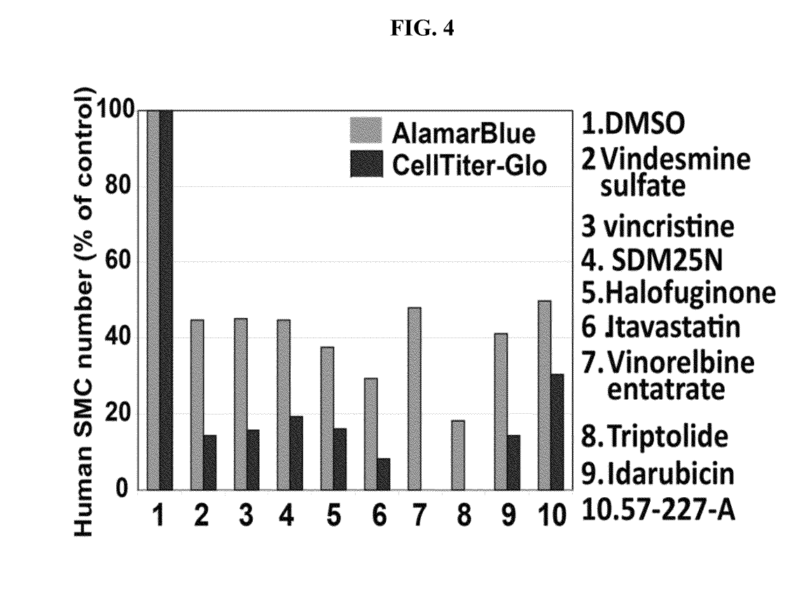

Among 447 drugs, 11 drugs inhibited human SMC proliferation greater than 50%, producing a hit rate of approximately 2.5% (FIG. 3). It is anticipated that the drugs providing more than 50% SMC inhibition will have a high likelihood of inhibiting intimal hyperplasia. The orthogonal CellTiter-Glo® luminescent cell viability assay has been used to confirm the hits from this pilot screen. The same plates in the pilot screen were subjected to CellTiter-Glo® assay after removal of AlamarBlue® followed by a gentle wash. As shown in FIG. 4, the CellTiter-Glo® assay produced a pattern of inhibition that was similar to AlamarBlue®.

### Example 3

### Endothelial Cell Proliferation Assay

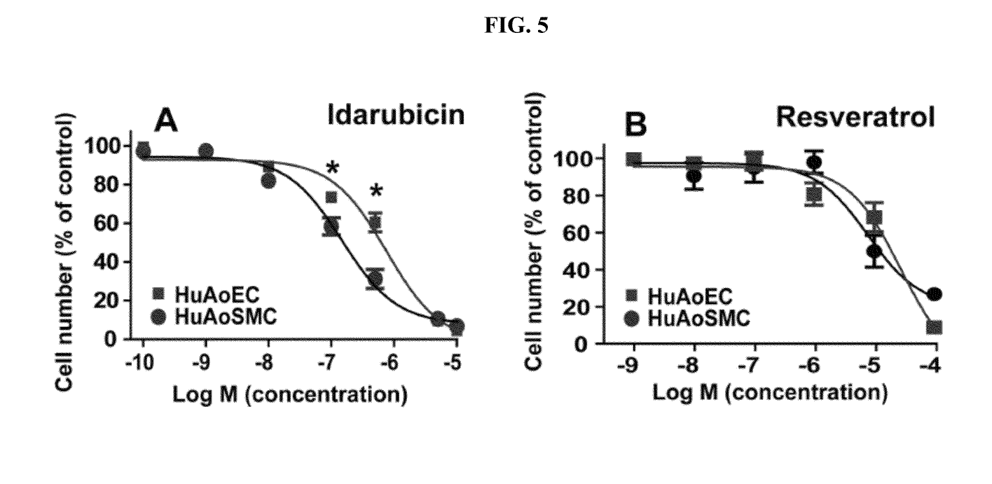

A counter-screen of compounds affecting human EC proliferation further identified idarubicin as a compound that preferentially inhibits SMCs over ECs. Confirming this result, dose response assays have shown that in a concentration range of approximately 50 nM to 1000 nM, idarubicin impedes SMC proliferation to a significantly greater extent than ECs (FIG. 5). Moreover, local application of idarubicin in the rat balloon angioplasty model reduced intimal hyperplasia by 70% but had no substantial impairment on the re-growth of denuded endothelial layer as demonstrated by CD31 antibody staining

These data suggest that idarubicin, an FDA-approved drug used for treatment of leukemia, is a selective anti-restenotic candidate drug for impeding intimal hyperplasia with only a minimal effect on re-endothelialization. Given that most of the conventional drugs that inhibit SMC proliferation also strongly inhibit the growth of ECs, idarubicin has improved properties and could be uniquely suited for in vivo developing methods of suppressing or preventing intimal hyperplasia.

### Example 4

### Preliminary Studies Using Rat Carotid Angioplasty Model

Resveratrol and halofuginone, two compounds that have been shown to inhibit SMC growth in our cell assays, were used as positive controls to demonstrate the utility of the rat carotid angioplasty in vivo model. Balloon injury of the left common carotid artery was performed in Male Sprague-Dawley rats (300-350 g) according to the method described previously (Kundi et al., Cardiovasc Res. 84(2):326-35 (2009). Briefly, after induction of anesthesia with isoflurane, a 2F balloon catheter was inserted through the left external carotid artery into the common carotid and insufflated three times at 2 atm of pressure. The external carotid artery was then ligated, and blood flow was resumed through the common and internal carotid arteries. For perivascular drug delivery, resveratrol (500 μg) or halofuginone (30 μg) or DMSO dissolved in 200 μL of 25% F127 pluronic gel (Sigma-Aldrich) in PBS was evenly applied, immediately after reestablishment of flow, around the injured segment of the carotid artery (5 animals in each group). The pluronic gel is a biodegradable polymer that is soluble in water at 4° C. but becomes a gel when in contact with tissues at 37° C. (Ji et al., Circ. Res. 100:1579-88 (2007)). Rats were euthanized 14 days after injury, and the common carotid arteries were collected and fixed in 4% paraformaldehyde overnight for embedding in paraffin.

For morphometric analysis of intimal hyperplasia, ten evenly spaced sections through each injured carotid artery were stained using routine hematoxylin and eosin (H&E) and images were collected with light microscopy. Intimal and medial areas and circumference were determined by measuring the internal elastic lamina (IEE) and external elastic lamina (EEL) for each section using the ImageJ software (National Institutes of Health). Kundi et al., Cardiovasc. Res. 84:326-35 (2009); Kingston et al., Circulation 108:2819-25 (2003). The difference in intimal hyperplasia between the compound treatment group and the control group was analyzed with the Student t-test (P<0.05).

To mark endothelial cells, immunohistochemistry using an anti-CD31 antibody was performed on rat carotid sections based on our published methods. Suwanabol et al., Am J Physiol Heart Circ Physiol. 302:H2211-19 (2012); Kundi et al., Cardiovasc. Res. 84:326-35 (2009); Si et al., Arterioscler. Thromb. Vasc. Biol. 32:943-54 (2012). Briefly, the carotid sections were incubated with the anti-CD31 antibody (Santa Cruz Biotechnology) overnight at 4° C., followed by rhodamine-conjugated secondary antibody (DAKO) for 60 minutes at room temperature. Antibody controls included species-matched IgG. Images were taken with a fluorescence microscope.

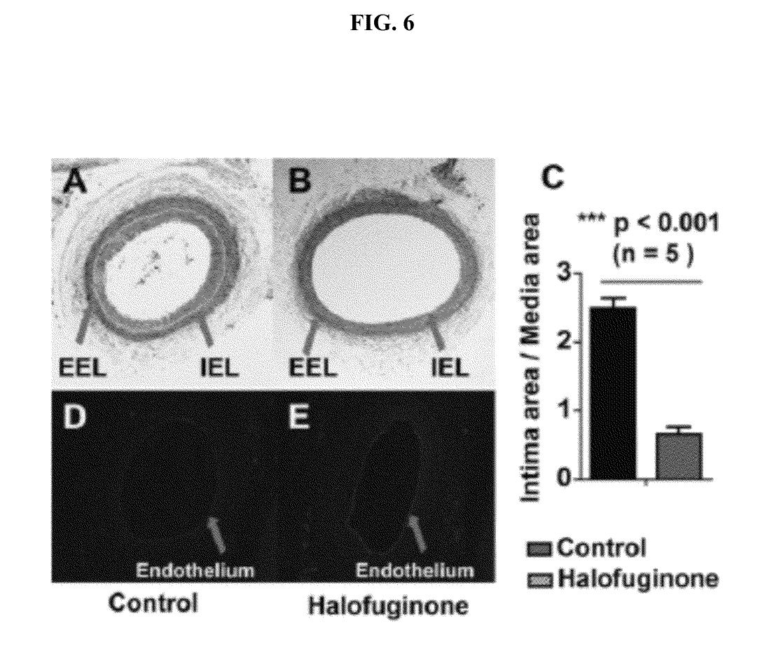

Using the rat angioplasty model and perivascular resveratrol delivery, 84% inhibition of the intima/media ratio was achieved. The intima/media ratio is a commonly used measure for intimal hyperplasia. Suwanabol et al., Am J Physiol Heart Circ Physiol. 302:H2211-19 (2012); Kundi et al., Cardiovasc. Res. 84:326-35 (2009); Kingston et al., Circulation 108:2819-25 (2003). Halofuginone periadventitially applied to the injured artery reduced intimal hyperplasia by 74% (FIG. 6C). CD31 immunostaining is often used as a marker to assess re-endothelilization. Takamiya et al., Arterioscler Thromb Vasc Biol 26:751-757 (2006); Yang et al., Intl. J. Exp. Cell. Physiol. Biochem. Pharmacol. 26:441-448 (2010). The data show that while halofuginone effectively reduced intimal hyperplasia, re-endothelilization of the injured vessel was not affected (see FIGS. 6D-E; CD31 antibody staining)

### Example 5

### Effect of Idarubicin in an In Vivo Rat Carotid Angioplasty Model

Balloon angioplasty was performed in the left common carotid artery of male Sprague-Dawley rats. Immediately after resuming the blood flow, idarubicin (110 μg) or DMSO control dissolved in 300 μL of 25% F127 pluronic gel was applied around the injured artery. At 14 days after injury, arteries were collected and cross-sections were prepared and H&E stained. Intimal and medial areas, and circumference were determined by measuring the internal elastic lamina (IEE) and external elastic lamina (EEL) for each section using the ImageJ software. The difference in intimal hyperplasia (ratio of intima to media) between the idarubicin treatment group and the control group was analyzed with the Student t-test (P<0.05). Immunostaining of CD31, an endothelial cell marker, was performed on the sections to assess re-endothelialization.

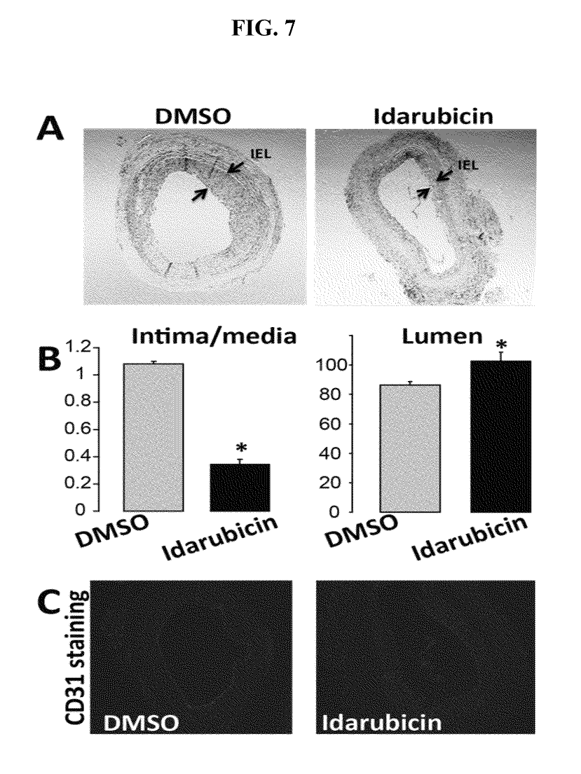

As shown in FIG. 7, the data (n=5 animals) show that while idarubicin effectively reduced intimal hyperplasia (A and B, intima/media), the integrity of the endothelial layer (re-endothelialization) of the injured vessel was not affected (C) as determined by CD31 immunostaining

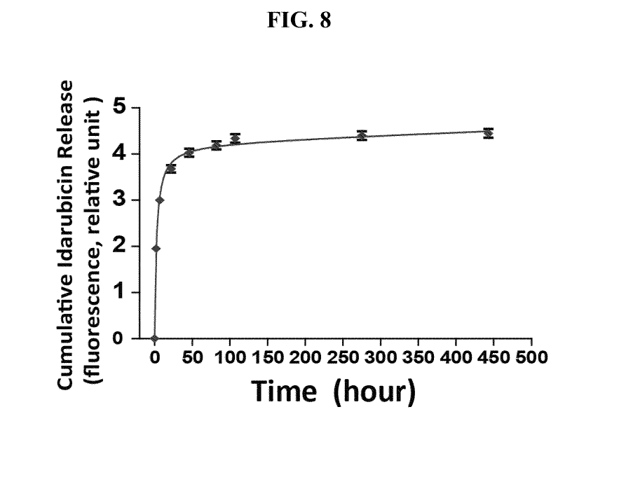

Three doses have been tested in vivo: 1 mg/mL (in pluronic gel), 0.5 mg/mL (FIG. 8), and 0.2 mg/mL. The highest dose caused slight cytotoxicity (e.g., small spots of necrosis in the surrounding tissues). The medium dose (0.5 mg/mL) and even the lowest dose (0.2 mg/mL, data not shown) inhibited intimal hyperplasia >70% without showing cytotoxicity, indicating that there is a potential to lower the effective drug dose substantially.

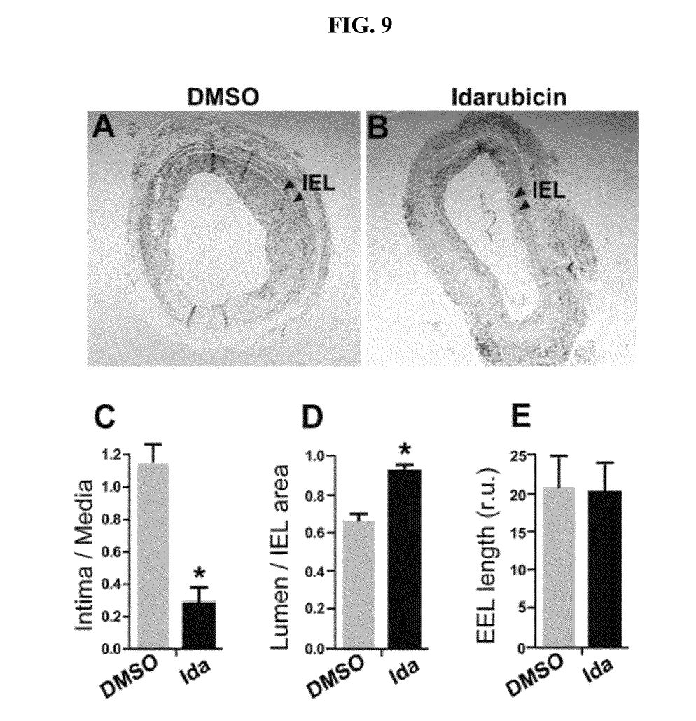

Drug-eluting stents or balloons are often used in clinics to deliver drugs for treating atherosclerosis or restenosis. Poly(lactic-co-glycolic acid) (PLGA) is an FDA-approved biodegradable polymer that is widely used for stent coating. Idarubicin was incorporated into the PLGA membrane to mimic a stent coating, and its release kinetics in PBS buffer were measured using idarubicin fluorescence. As shown in FIG. 9, after an initial phase of fast release in the first two days (which is characteristic of drug-release from polymer coatings), steady idarubicin release from the polymer continued beyond 18 days.

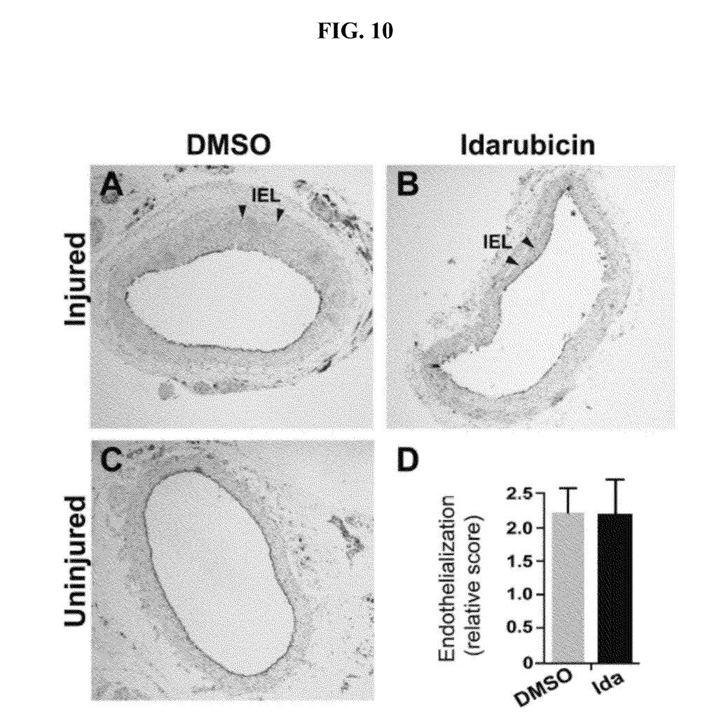

In our in vitro experiments, idarubicin differentially inhibited SMC proliferation with a lesser effect on ECs (FIG. 5). With this in mind, it was further explored whether idarubicin could spare the endothelial layer while attenuating the growth of the neointima in vivo. Using the carotid artery sections collected on day 14 following angioplasty, we performed immunostaining for CD31 (FIGS. 10A-C), a commonly used marker for assessment of the endothelium, in a rat carotid injury model at 14 days. Briefly, a goat anti-CD31 primary antibody (R&D Sytems, 1:150) was incubated with the sections for 1 hour followed by an incubation with a biotinylated rabbit-anti-goat secondary antibody for 30 minutes. Immunostaining of CD31 was then visualized by using streptavidin-HRP and DAB. Re-endothelialization was quantified following previously published methods (Tian et al., Journal of Endovascular Therapy 13:616-629 (2006); Brown et al., Arterioscler Thromb Vasc Biol 30:2150-2155 (2010)). Briefly, the percentage of the luminal perimeter that stained for CD31 versus total perimeter was measured using NIH Image J. Re-endothelialization was then scored from 1 to 5 (1: >20%; 2: 20 to 40%; 3: 40 to 60%; 4: 60 to 80%; 5: 80%-100%) and the scores were averaged with the data from 5 rats (6 sections per rat) in each treatment group.

Quantification of CD31 staining revealed an 80% reduction of intimal hyperplasia and a 45% increase of lumen size with no significant effect on re-endothelialization. Thus, the extent of re-endothelialization observed in idarubicin-treated arteries was similar to that of vehicle-treated arteries (FIG. 10D). These data suggest that idarubicin, as a potent inhibitor of SMC proliferation and intimal hyperplasia, does not impose a significant inhibitory effect on the endothelial recovery after angioplasty denudation. Importantly, the data presented herein demonstrate that the leukemia drug idarubicin is highly effective in reducing angioplasty-caused intimal hyperplasia (or restenosis) resulting from angioplasty but has minimal effects on re-endothelialization. As one of few agents that have been shown to preferentially inhibit the proliferation of SMCs versus ECs, idarubicin is a promising candidate second-generation drug for treating restenotic disease.

### Prophetic Example

### Therapeutic Anti-Restenosis Device and Methods of Use

The following prophetic example, which is provided for illustrative purposes only and not to limit the invention, illustrates preparation and use of an implantable device to treat or prevent restenosis.

A perivascular drug delivery composition is prepared by dissolving 0.08 mg/mL idarubicin and 500 μg resveratrol or 30 μg halofuginone in 200 μL of 25% F127 pluronic gel. The composition is applied to a stent to form a drug layer on the stent. A spray apparatus can be used to apply the idarubicin composition to the stent. Alternatively, the stent is dipped in the idarubicin composition to form the drug layer. Optionally, the stent is further coated in one or more additional layers such as a primer layer and a topcoat layer.

The idarubicin-coated stent is then used in a method to treat or prevent restenosis in a patient who has received a localized vascular injury or who is at risk of vascular occlusion. The localized vascular injury can result from an angiographic procedure. Generally, an angiographic procedure includes placement of a balloon catheter at an occlusion site, and a distal-end balloon is inflated and deflated one or more times to force the occluded vessel open. This vessel expansion can result in localized injury, which often causes inflammation, cell proliferation, and reocclusion over time. Following a balloon angioplasty, therefore, an idarubicin-coated stent is positioned at or near the site of vascular injury. If the idarubicin-coated stent has an expandable member, the member is expanded at or near the site of vascular injury until the stent contacts the vessel walls. Once deployed at the site, the idarubicin-coated stent releases the active compounds to cells lining the vascular site, thereby preferentially inhibiting smooth muscle cell proliferation but having minimal effects on re-endothelialization at the site of vascular injury.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

We claim:

1. A medical device comprising a formulation comprising between about 0.08 mg/mL and about 0.4 mg/mL idarubicin.

2. The device of claim 1, wherein the formulation further comprises at least one of rapamycin, resveratrol, and halofuginone.

3. The device of claim 1, wherein the formulation further comprises a pharmaceutically acceptable carrier.

4. The device of claim 3, wherein the pharmaceutically acceptable carrier is a pluronic gel.

5. The device of claim 1, wherein the device comprises a drug-eluting stent.

6. The device of claim 1, wherein the device further comprises an expandable member having a first diameter for insertion into a vessel and a second diameter for making contact to walls of the vessel.

7. The device of claim 6, wherein at least a portion of the outer surface of the expandable member comprises the idarubicin formulation.

8. The device of claim 6, wherein the expandable member comprises a balloon.

9. A method for treating or preventing restenosis in a subject in need thereof, the method comprising, following a vascular intervention that exposes the lumen of a blood vessel of the subject, providing to the lumen an effective amount of a compound capable of selectively decreasing smooth muscle cell proliferation without a substantial decrease in endothelial cell proliferation, wherein providing the effective amount of the compound treats or prevents restenosis in the blood vessel.

10. The method of claim 9, wherein the compound is idarubicin or an analog thereof.

11. The method of claim 10, wherein the effective amount of idarubicin or an analog thereof is between about 0.08 mg/mL and about 0.4 mg/mL.

12. The method of claim 10, wherein idarubicin or an analog thereof is provided to the lumen in a formulation comprising at least 0.08 mg/mL idarubicin or an analog thereof.

13. The method of claim 12, wherein the idarubicin formulation further comprises at least one of rapamycin, resveratrol, and halofuginone.

14. The method of claim 9, wherein the compound is provided to the lumen using a medical device.

15. The method of claim 14, wherein the medical device comprises a drug-eluting stent.

16. The method of claim 9, wherein the vascular intervention is selected from the group consisting of an angioplasty, an atherectomy, a bypass surgical procedure, placement of a non-drug eluting stent, and placement of a drug-eluting stent.

17. The method of claim 9, wherein treating or preventing restenosis comprises reducing intimal hyperplasia.

18. A method for treating or preventing a vascular disease in a subject in need thereof, the method comprising providing to the lumen of a blood vessel of the subject an effective amount of a compound capable of selectively decreasing smooth muscle cell proliferation without a substantial decrease in endothelial cell proliferation, whereby vascular disease in the subject is treated or prevented.

19. The method of claim 18, wherein the vascular disease is atherosclerosis.

20. The method of claim 18, wherein the compound is idarubicin or an analog thereof.

21. The method of claim 20, wherein the effective amount of idarubicin or an analog thereof is between about 0.08 mg/mL and about 0.4 mg/mL.

22. The method of claim 20, wherein idarubicin or analog thereof is provided to the lumen in a formulation comprising at least 0.08 mg/mL idarubicin.

23. The method of claim 20, wherein the idarubicin formulation further comprises at least one of rapamycin, resveratrol, and halofuginone.

24. The method of claim 18, wherein the compound is provided to the lumen using a medical device.

25. The method of claim 24, wherein the medical device comprises a drug-eluting stent.

26. The method of claim 18, wherein the subject has undergone or will undergo a vascular intervention.

27. The method of claim 26, wherein the vascular intervention is selected from the group consisting of an angioplasty, an atherectomy, a bypass surgical procedure, placement of a non-drug eluting stent, and placement of a drug-eluting stent.

28. The method of claim 18, wherein treating or preventing a vascular disease comprises reducing intimal hyperplasia.
